(19) United States
(12) Patent Application Publication
(10) Pub. No.: US 2013/0047347 A1
(43) Pub. Date: Feb. 28, 2013

(54) Depilatory Method and Kit

(71) Applicants: Charles Robert SMITH, Swindon (GB); Stuart Andrew HEWLINS, Woking (GB); Michael John GOFFE, Egham (GB)

(72) Inventors: Charles Robert SMITH, Swindon (GB); Stuart Andrew HEWLINS, Woking (GB); Michael John GOFFE, Egham (GB)

(21) Appl. No.: 13/593,763

(22) Filed: Aug. 24, 2012

Related U.S. Application Data
(60) Provisional application No. 61/526,701, filed on Aug. 24, 2011.

(51) Int. Cl. A61K 8/81; A61Q 9/04; A61K 8/46

(52) U.S. Cl. 8/161

(57) ABSTRACT

A method of removing hair from skin is provided by the steps of providing a protective composition, wherein the protective composition comprises at least 1% of a hydrophobic polymer by weight of the protective composition and wherein the protective composition reduces the penetration of thioglycolic acid by at least 45%, as measured using the Franz Cell Method; and, providing a depilatory composition comprising an effective amount of a keratin reducing agent.

### CROSS-REFERENCE TO RELATED APPLICATION

This application claims the benefit of U.S. Provisional Application No. 61/526,701, filed 24 Aug. 2011.

### FIELD OF THE INVENTION

The present invention relates to a method and kit for depilation.

### BACKGROUND OF THE INVENTION

Depilatory compositions are cosmetic hair removal formulations. They comprise keratin reducing agents, which attack the disulphide bonds in hair to weaken it, such that subsequent gentle scraping and/or wiping completes severance of the hair from the skin and effects hair removal. Commercially, the most common keratin reducing agents are thioglycolates, which are typically formulated at high pH. An unwanted side effect of chemical depilation is that the depilatory composition comes into contact with and must have a relatively long residence time on skin to achieve effective hair removal and this long residence time combined with the alkaline conditions needed for effective hair removal may give rise to skin irritation.

The above problem has been recognized in the art. Reference is made to U.S. Pat. No. 4,401,663 and U.S. Pat. No. 4,424,205 the disclosures of which are similar to one another. These documents teach to use certain compounds as topical analgesics and an example given of a situation in which it is suggested to use such materials is to reduce depilatory irritation. The carrier formulas disclosed to be suitable for formulation with the analgesics include lotions and creams.

Reference is also made to US 2004/0219118, which discloses treatment with a “lipophilic” material before application of a thioglycolate-based reactive depilatory compostion. Lipophilic materials exemplified in this patent application are oils, such as mineral oil. As shown hereinbelow, the present applicants have tested a range of lipophilic materials to determine their ability to prevent thioglycolate penetration and, thereby, their ability to reduce or prevent skin irritation Applicants have surprisingly found that oils, such as mineral oil, have no or a low ability to prevent thioglycolate penetration to the skin. There thus exists a need to develop a pre-treatment composition which better reduces skin irritation.

### SUMMARY OF THE INVENTION

According to a first aspect of the invention, a depilatory kit is provided, comprising:
(a) a protective composition, wherein the protective composition comprises at least 1% of a hydrophobic polymer by weight of the protective composition and wherein the protective composition reduces the penetration of thioglycolic acid by at least 45%, as measured using the Franz Cell Method;
(b) a depilatory composition comprising an effective amount of a keratin reducing agent.

According to a second aspect of the invention, a method of removing hair from skin is provided, comprising the steps of:
(a) applying a protective composition defined in the first aspect of the invention to an area of skin on which unwanted hair is growing;
(b) applying a depilatory composition to the area of skin to which the protective composition has been applied, the depilatory composition comprising a keratin reducing agent.

### BRIEF DESCRIPTION OF THE DRAWINGS

FIG. 1. is a schematic view of a Franz Cell apparatus.

### DETAILED DESCRIPTION OF THE INVENTION

The protective composition used in the method and comprised within the kit according to the invention comprises at least 1% of a hydrophobic polymer and reduces the penetration of thioglycolic acid by at least 45%, as measured using the Franz Cell Method. A reduction of thioglycolic acid penetration of 45% or more according to the Franz Cell test method may be shown to correlate to a significant and user-noticeable reduction in irritation.

The protective composition used in the method and comprised within the kit according to the invention advantageously comprises from 1% to 50%, preferably from 2% to 30%, more preferably from 3% to 20% of the hydrophobic polymer by weight of the protective composition.

According to the invention, the hydrophobic polymer is advantageously a copolymer, the copolymer comprising at least 50%, preferably from 50% to 95%, more preferably from 60% to 90% by weight of the copolymer of monomers with hydrophobic side-groups. The side-groups comprise from 8 to 40, preferably from 8 to 30 carbon atoms. Advantageously, the hydrophobic side-groups are straight chain alkyl groups. These side-groups render the copolymer hydrophobic in nature.

Advantageously, the carbon-carbon bonds within the hydrophobic side-groups have a degree of unsaturation of less than 50%, preferably less than 25% and are more preferably fully saturated. The percentage degree of unsaturation (% DU) may be calculated using the following formula:

$$\% DU = ( 1 + {\sum }^{ } \frac{\begin{matrix}( N ° ( C - C ) Double Bonds ) + \\ ( 2 \times N ° ( C - C ) Triple Bonds )\end{matrix}}{N ° Carbons} ) \times 100$$

wherein:
the term “N° (C—C) Double Bonds” refers to the number of double bonds with the hydrophobic side-chain
the term “N° (C—C) Triple Bonds” refers to the number of triple bonds with the hydrophobic side-chain
the term “N° Carbons” refers to the number of carbon atoms with the hydrophobic side-chain

According to one embodiment according to the invention, the copolymer comprises monomers having formula (monomer A):

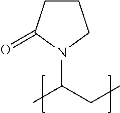

and monomers having formula (monomer B):

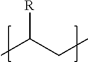

where R is a hydrophobic side-group comprising a straight alkyl chain having from 12 to 30 carbon atoms and wherein the copolymer comprises from 50% to 99%, more preferably 50% to 90% by weight of monomer B with the remainder being monomer A.

According to another embodiment according to the invention, the copolymer comprises monomers having formula (monomer D):

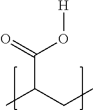

and monomers having formula (monomer E):

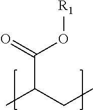

where R₁ is a hydrophobic side-group comprising a straight alkyl chain having from 10 to 30 carbon atoms; and monomers having formula (monomer F):

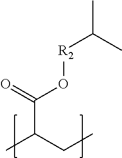

where R₂ is a hydrophobic side-group comprising a straight alkyl chain having from 4 to 26 carbon atoms, wherein the copolymer comprises from 50% to 99%, more preferably 50% to 90% by weight of the combination of monomers E and F, with the remainder being monomer D.

The protective composition may comprise a single phase or more than one phase.

If the protective composition comprises a single phase, then, given the hydrophobic nature of the hydrophobic polymer, the protective composition will essentially a single hydrophobic phase (although it may comprise a few cosmetic adjuncts dispersed within it, which are not hydrophobic). In this case, the single phase protective composition may additionally comprise other hydrophobic materials, such as oils, waxes and triglycerides (fats).

If the protective composition comprises more than one phase, then it may be in the form of an emulsion, which may have a hydrophilic continuous phase and a hydrophobic dispersed phase or a hydrophobic continuous phase and a hydrophilic dispersed phase. The hydrophobic polymer is comprised within the hydrophobic phase, whether that phase is the dispersed or the continuous phase. As in the case in which the protective composition comprises a single phase, the hydrophobic phase may comprise other materials mixed with the hydrophobic polymer, such as oils, waxes, triglycerides (fats), oil soluble skin active agents and mixtures thereof.

Oils which may be mixed with the hydrophobic polymer to form the hydrophobic phase, whether part of a single or multi-phase composition, include natural oil, synthetic oil, silicone oil and mixtures thereof.

Waxes which may be mixed with the hydrophobic polymer to form the hydrophobic phase, whether part of a single or multi-phase composition, include natural wax, synthetic wax, silicone wax and mixtures thereof.

Triglycerides which may be mixed with the hydrophobic polymer to form the hydrophobic phase, whether part of a single or multi-phase composition, have the following formula:

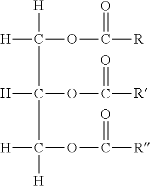

wherein R, R′ and R″ may be the same as or different from one or both of the others, wherein each of R, R′ and R″ is a fatty acid.

Oil soluble skin active agents which may be mixed with the hydrophobic polymer to form the hydrophobic phase, whether part of a single or multi-phase composition, oil soluble vitamins, such as vitamin E derivatives, including vitamin E acetate and tocopherol nicotinate; oil-soluble vitamin A derivatives, such as retinyl palmitate; lanolin; ceramides; sterols and sterol esters; salicylic acid; camphor; eucalyptol; essential oils and mixtures thereof.

The protective composition used in the method and comprised within the kit according to the invention may include small quantities of further ingredients such as, but not limited to metal oxides, organic and inorganic dyes, lakes, micas, flavourings, perfumes and mixtures thereof.

If the protective composition used in the method and comprised within the kit according to the invention comprises an emulsion, then it advantageously comprises from 4.9% to 89.9%, preferably from 10% to 75%, more preferably from 25% to 65% hydrophilic phase by weight of the protective composition.

The hydrophilic phase typically comprises water, but need not. In addition to or as an alternative to water, the hydrophilic dispersed phase may comprise hydrophilic materials, such as polyhydric alcohols, ethoxylated and propoxylated polyols, polysaccharides, and mixtures thereof. Suitable polyhydric alcohols (polyols) include, but are not limited to, butylene glycol, hexylene glycol, ethoxydiglycol, dipropylene glycol, phenyl ethyl alcohol, glycerin, 1,3-butanediol, 1,2-propanediol, isoprene glycol, sorbitol, polyethylene glycol, polypropylene glycol and mixtures thereof. Preferred polyols include glycerin, propylene glycol, panthenol and mixtures thereof.

Additionally, the hydrophilic phase may comprise other polar solvents, such as alcohols, ketones and mixtures thereof. Examples of suitable polar solvents include phenyl ethyl alcohol, ethanol, isopropyl alcohol and mixtures thereof.

The hydrophilic phase may additionally comprise hydrophilic skin active agents such as, but not limited to, vitamins, including hydrophilic ascorbic acid compounds and vitamin B3 compounds; azelaic acid; gallic acid and its derivatives; N-acetyl glucosamine; panthenol and mixtures thereof.

If the protective composition used in the method and comprised within the kit according to the invention is an emulsion then it will comprise an appropriate emulsifier selected by the skilled person, according to the type of emulsion in question. The protective composition will typically comprise from 0.1% to 10%, and preferably from 0.1% to 5% emulsifier by weight of the protective composition. The emulsifier may be nonionic, anionic or cationic. Suitable emulsifiers are disclosed in McCutcheon's Detergents and Emulsifiers, North American Edition, pages 317-324 (1986).

Any depilatory composition comprising a suitable keratin reducing agent may be used in the present method and included in the present kit. Non-limiting examples of suitable keratin reducing agents include: sulphide salts such as Li₂S, Na₂S, K₂S, MgS, CaS, SrS or BaS, hydrogen sulphide salts such as NaSH or KSH; thioglycol; thioglycerol; thioglycolamide; thioglycolhydrazide; thioglycolic acid; thioglycolate salts (such as potassium thioglycolate, calcium thioglycolate, ammonium thioglycolate, diammonium dithioglycolate, glyceryl monothioglycolate, or monoethanolamine thioglycolate); thiosalicylic acid; thiomalic acid; ammonium thiolactate; monoethanolamine thiolactate; dithioerythritol; 2-mercaptopropionic acid; 1,3-dithiopropanol; glutathione; dithiothreitol; cysteine; homocysteine; N-acetyl-L-cysteine and cysteamine. Advantageously, the keratin reducing agent is comprised within the depilatory composition in an amount from 0.3% to 20%, preferably from 0.8% to 15%, more preferably from 1% to 10% by weight of the depilatory composition.

Advantageously, the depilatory composition may comprise at least one thioglycolate salt or thioglycollic acid acting as a hair removal agent when the depilatory composition is applied to unwanted hair. Preferably, the depilatory composition comprises sodium, potassium, magnesium, calcium, beryllium, strontium, zinc, monoethanolamine, ammonium, tetralkylammonium, imidazolium, pyridinium, phosphonium or glyceryl thioglycolate salts, or mixtures thereof, which may include dianion forms of thioglycolate. More preferably, the depilatory composition comprises at least one of sodium, potassium, magnesium or calcium thioglycolate, or mixtures thereof. Even more preferably the depilatory composition comprises potassium or calcium thioglycolate, or mixtures thereof.

The pH of the depilatory composition may advantageously be in the range of from 6 to 13.8, preferably from greater than 7 to 13, more preferably from 9 to 12.9, even more preferably from 10 to 12.8, even more preferably still from 12 to 12.75 and yet more preferably from 12.3 to 12.6 to improve the efficacy of the active ingredient. The depilatory composition may, in a preferred embodiment, comprise at least one base to control the pH. Preferably, the depilatory composition comprises potassium hydroxide; sodium hydroxide; lithium hydroxide; calcium hydroxide; barium hydroxide; caesium hydroxide; sodium hydroxide; ammonium hydroxide; strontium hydroxide; rubidium hydroxide; magnesium hydroxide; zinc hydroxide; sodium carbonate; pyridine; ammonia; alkanolamides (including monoethanolamine, diethanolamine, triethanolamine), phosphates (including tetrasodium phosphate), arginine or mixtures thereof. More preferably, the depilatory composition comprises at least one buffering base, even more preferably the depilatory composition comprises calcium hydroxide, magnesium hydroxide; barium hydroxide; strontium hydroxide; zinc hydroxide; arginine or mixtures thereof. Still more preferably the depilatory composition comprises calcium hydroxide; magnesium hydroxide, zinc hydroxide, sodium hydroxide, potassium hydroxide or mixtures thereof. Even more preferably still, the depilatory composition comprises calcium hydroxide, sodium hydroxide or mixtures thereof.

In an advantageous embodiment, the base is present at a concentration of from 0.1% to 10.0%, more preferably from 0.5% to 8.0% and even more preferably from 1.0% to 5.0%, by weight of the depilatory composition.

The concentration of water in the depilatory composition is preferably at least 40%, more preferably from 50% to 98%, even more preferably from 60% to 95% and even more preferably still from 70% to 90%, by weight of the depilatory composition.

The depilatory composition may optionally comprise a thickening agent. A representative but not exhaustive list can be found in “The Encyclopaedia of Polymers and Thickeners for Cosmetics” compiled and edited by Robert Y. Lochhead, PhD and William R. Fron, Department of Polymer Science, University of Southern Mississippi. Exemplary classes of thickening agents include gums, carbomers, polymers and copolymers of acrylic acid, associated thickeners, layered silicates/clays and natural polymers (including polysaccharides). One or more thickening agents may be included in the aqueous depilatory composition. The thickening agent may be present at a level of from about 0.01% to about 20%, preferably from about 0.1% to about 10% by weight of the depilatory composition.

The depilatory composition may also include other skin care ingredients such as conditioning agents selected from the group consisting of humectants, moisturizers, or skin conditioners (including mineral oil; almond oil; chamomile oil; jojoba oil; avocado oil; shea butter, niacinamide and glycerine); skin rejuvenation compositions (for example targeted for fine lines, wrinkles and uneven skin tone, including retinoids), cosmetic compositions; anti-inflammatory agents (including corticosteroids); anti-oxidants (including flavonoids) radical scavengers; sunscreen agents; skin cooling or warming agents and the like. The depilatory composition may comprise one or more skin care ingredients present in an amount of from about 0.001% to about 10%, more preferably from about 0.01% to about 7%, and even more preferably from about 0.025% to about 5%, by weight of the depilatory composition.

An accelerant may be employed in the depilatory composition. This optional component accelerates the rate of depilatory action of the depilatory agent. Suitable accelerants include, but are not limited to, urea; thiourea; dimethyl isosorbide; arginine salts; ethoxydiglycol; propylene glycol and methylpropyldiol. The accelerant may be present in a concentration range of from 0.5% to 10%, more preferably from 2% to 8% and even more preferably from 2% to 5% by weight of the depilatory composition.

The depilatory composition may further comprise components known, conventionally used, or otherwise effective for use in cosmetic compositions, such as dyes; pigments (including ultra marines and talc); anionic, cationic, non-ionic and/or amphoteric or zwitterionic surfactants, polymers (including hydrophobically modified polymers); dispersing agents; solvents; lubricants; fragrances; preservatives; chelants, proteins and derivatives thereof, plant materials (e.g. aloe, chamomile and henna extracts); silicones (volatile or non-volatile, modified or non-modified); film-forming agents; film forming promoters and mixtures thereof.

The depilatory composition may be formulated in any common delivery form, such as a cream or lotion. Alternatively, it may be delivered on a substrate, such as a thin film of depilatory composition coated onto the substrate. The substrate may be configured in any suitable form, such as a strip, mask or patch.

In addition to the protective composition and the depilatory composition, the kit according to the second aspect of the invention may comprise one or more of:
(a) A make-up removal composition and/or a make-up removal wipe;
(b) Means for removal of the protective composition and the depilatory composition following use, which means may comprise one or more of a tool, such as a scraper or a spatula; or a wipe;
(c) A post-treatment composition skin care composition to be applied to the area of skin from which hair has been removed. Such a post-treatment skin care composition may comprise ingredients to promote skin conditioning; moisturizers, skin rejuvenation compositions (targeted for fine lines, wrinkles and uneven skin tone, for example), cosmetic compositions (e.g., foundation, rouge), sunscreens and the like. The post-treatment skin care composition may be leave-on or a rinse-off composition.
(d) Instructions regarding how to use the various elements of the kit, which instructions may comprise one or more elements of the method as defined herein.

Prior to applying the method or using the kit according to the present invention, a user should advantageously remove all make-up from the skin, to ensure good adherence and effective application of both the protective composition and the depilatory composition.

The method according to the first aspect of the invention comprises the step of applying the above-defined protective composition to an area of skin on which unwanted hair is growing, which may be located on any part of the human body.

Advantageously, the protective composition is not just applied to the area to be depilated, but also to an immediately juxtaposing area thereabout (that is, the protective composition is applied to an area of skin which is greater than just the area which is to be depilated).

Advantageously, the user will apply from 0.3-2 mg of protective composition per square centimetre of skin, preferably from 0.4-1.3 mg/cm², more preferably from 0.4 to 1 mg/cm².

Following application, the protective composition is advantageously massaged into the skin Preferably, massaging is effected for at least 10 seconds, and, more preferably, massaging is effected as a circular motion. Without wishing to be bound by theory, it is believed that the protective composition may trap hair within it thereby shielding it from the to-be-applied depilatory composition; massaging may help to release the hairs from the skin and ensure improved access thereto by the depilatory composition.

The method according to the first aspect of the invention comprises the subsequent step of applying the above-defined depilatory composition to an area of skin on which unwanted hair is growing and to which protective composition has already been applied. Advantageously, the user will apply a layer of depilatory composition which is from 0.1 mm to 5 mm, preferably from 0.3 to 3 mm, more preferably from 0.5 to 2 mm in thickness.

Subsequently, according to the method of the first aspect of the invention, the depilatory composition is advantageously left in place for at least 1 minute, preferably from 1 to 10 minutes, more preferably from 3 to 10 minutes, depending on the thickness of the hair and the hair removal efficacy of the depilatory composition (which, in turn, is dependent upon the concentration of keratin reducing agent in the depilatory composition).

Subsequently, according to the method of the first aspect of the invention, the protective composition and the depilatory composition are advantageously removed. This may be achieved using one or more of a cotton wool ball, pad or wand, a tissue, a cloth, or a tool, such as a spatula or a scraper. Advantageously, the skin from which hair has been removed is then rinsed with water.

In an advantageous subsequent step, a post-treatment skin care composition may be applied to the area of skin from which hair has been removed. Such a post-treatment skin care composition may comprise ingredients to promote skin conditioning; moisturizers, skin rejuvenation compositions (targeted for fine lines, wrinkles and uneven skin tone, for example), cosmetic compositions (e.g., foundation, rouge), sunscreens and the like. The post-treatment skin care composition may be leave-on or a rinse-off composition.

### Franz Cell Method

### Principle and Scope:

This method is applicable for using Franz cell apparatus for the in-vitro assessment of penetration of thioglycolic acid (TGA) and its salts through a skin mimic after the application of a depilatory composition following pre-treatment with a protective composition.

Penetrated TGA is quantified using Reverse Phase High Performance (or Pressure) Liquid Chromatography (RP-HPLC) with external standard quantitation at 240 nm.

### Method

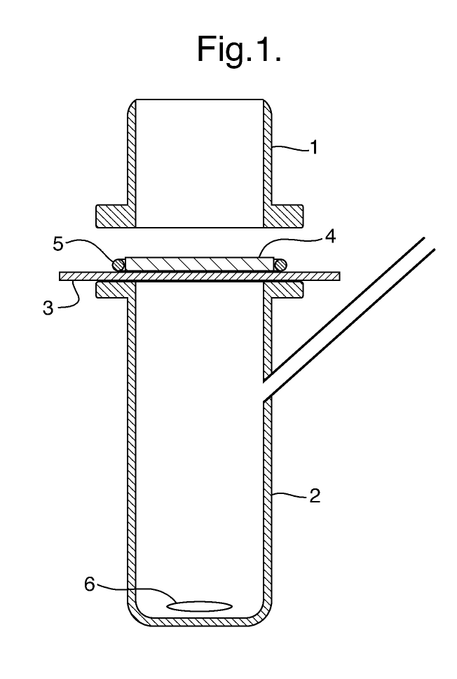

Reference is made to FIG. 1 and to the reference numerals therein:

1. Prepare the Vitro-Skin (IMS Vitro-Skin®, Catalogue number: P&G1013, made by IMS Inc., Portland, Me., USA) samples by cutting 8×6.2 cm segments and placing them textured side up on the racks into a hydration chamber (manufactured & sold by IMS) containing a 14.7% glycerol solution. The hydration chamber should be sealed and the vitro-skin left to hydrate at room temperature and a humidity of 80.4%±3.5% for 24 hours.
2. Prepare the receptor solution for the Franz-cell by mixing 1.90 ml formic acid (98% wt+Fluka, by Sigma Aldrich, or equivalent), 30 ml acetonitrile (RP-HPLC grade) and 968.1 ml water (RP-HPLC grade). Set up the static Franz cell (Permegear or equivalent, 15 mm diameter unjacketed cell with a 12 ml receptor volume) by clamping it in place over suitable stirrer plates (not shown) and add a small stirrer bar (6) to each cell, fill the receptor cell (2) to the brim with the required amount of receptor solution.
3. Once hydrated, remove a sheet of vitro-skin from the hydration chamber and lay textured side up on a clean flat surface then dose 100 μl (˜2 mg/cm²) of protective composition (not shown) onto the vitro-skin and spread evenly over the surface by rubbing for 30 seconds with a gloved finger.
4. Using a scalpel blade cut the vitro-skin segment (3) into two equal sections, each large enough to completely cover the top of the cell. Place the relevant size o-ring (5) (22 mm, for the specified Franz-cell) onto each section of the vitro skin and dose to 150 mg/cm² of depilatory composition (4) (“Veet Normal Skin Hair Removal Cream” or an equivalent (an equivalent being a composition comprising 3.7% wt thioglycolic acid)) into the centre then, using a glass rod, evenly spread the cream around the inside of the o-ring (5). Using tweezers pick up the vitro-skin segment and place the vitro-skin segment, depilatory and o-ring centrally over the receptor cell (2), place donor cell (1) over the top and clamp in place. Turn on stirrer plate and start 10 minute countdown timer. After 10 minutes; turn off stirrer and remove the clamp, donor cell (1) and vitro-skin segment and place the receptor solution in a suitable container for analysis.
5. A reference sample should also be run without protective composition treatment on the vitro-skin Remove a sheet of vitro-skin from the hydration chamber and lay textured side up on a clean flat surface. Repeat step 4 of the protocol to produce the reference sample.

### Sample Analysis

For RP-HPLC analysis, prepare a 50 mM Formic acid (98%+Fluka) solution and mix 970 ml of this solution with 30 ml acetonitrile (HPLC grade) to act as a mobile phase during the analysis.

A reference standard solution should be made with a concentration of Calcium Thioglycolate Trihydrate of 0.94 mg/ml.

Install a Waters Atlantis T3 3μm 4.6×50 mm column into the HPLC (although any silica-based C₁₈ reversed phase RP-HPLC column may be used), and ensure all solvent lines for the RP-HPLC are primed and free of leaks. Allow the mobile phase to circulate through the system for 25 minutes at 0.7 mL/Min in order to equilibrate the column. Detection of the thioglycolic acid is via UV spectroscopy.

The RP-HPLC conditions are as follows:

Injection volume: 20 μL

Mobile phase flow rate: 0.70 ml/min

Run time: 10 minutes

UV Detection wavelength: 240 nm

Column temperature: 35° C.

UV sampling rate: ≧5 per second

Retention time: Thioglycolic Acid ˜2.5 min

### Calculations:

Calculate the concentration of Thioglycolic Acid in the sample

$$concentration ( mg / ml ) = {weight}_{25} of std ( mg ) \times {purity}_{25} \times 3$$

Calculate the concentration of thioglycolic acid in the sample using the following formula:

concentration (mg/ml)=A/B×C×E/F

### Where,

### A=Peak Area of Thioglycolic Acid Sample

### B=Average Peak Area of Thioglycolic Acid Standard

C=Thioglycolic Acid final STD concentration in mg/ml (0.94 mg/ml)
E=Molecular weight of Thioglycolic acid (92.12 g/mol)
F=Molecular weight of Calcium thioglycolate (184.23 g/mol)

The efficacy of the barrier provided by the protective e composition (resistance to TGA penetration) can be calculated as a percentage decrease in TGA in the receptor solution:

$$\% reduction = \frac{concentration without barrier * - concentration with barrier *}{concentration without barrier *} \times 100$$

*protective composition

For example, if TGA in solution without protective composition=75 μg/ml and TGA in solution with barrier=15 μg/ml

$$\% reduction = \frac{75 - 60}{75} \times 100 = \underline{85 \%}$$

A reduction of TGA penetration of 45% or more is believed to correlate to a significant and user-noticeable reduction in irritation.

### EXAMPLES

| | | % Reduction in |
|---|---|---|
| | | Thioglycolic |
| | | Acid Penetration |
| | | According to the |
| Example | Protective Composition | Franz Cell Method |
| Inventive | 60% Mineral Oil | 100.0 |
| Example 1 | 40% Antaron WP660 | |
| | (Vinyl Pyrolidone/Triacontene | |
| | Copolymer) | |
| Inventive | 85% Mineral Oil | 94.0 |
| Example 2 | 15% Antaron V220F | |
| | (Alkylated Polyvinyl Pyrrolidone | |
| | with Eicosene 30/70 ratio) | |
| Inventive | 92% Hexamethyldisiloxane | 100.0 |
| Example 3 | 6% Acrylate terpolymer | |
| | (acrylic acid, stearyl methacrylate, | |
| | isooctyl methacrylate terpolymer) | |
| | 2% Trimethyl phenyl silsesquioxan | |
| Comparative | 100% mineral oil | 25.0 |
| Example 1 | | |
| Comparative | 100% Sunflower Seed oil | 10.8 |
| Example 2 | | |
| Comparative | 100% olive oil | 0.0 |
| Example 3 | | |

As demonstrated by the Franz Cell data, oils, on their own (see Comparative Examples 1, 2 and 3) provide little to no barrier to thioglycolic acid, whereas those comprising a hydrophobic polymer as defined herein present a dramatically superior barrier to penetration (see Inventive Examples 1-3).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A depilatory kit comprising:
(a) a protective composition, wherein the protective composition comprises at least about 1% of a hydrophobic polymer by weight of the protective composition and wherein the protective composition reduces the penetration of thioglycolic acid by at least about 45%, as measured using the Franz Cell Method;
(b) a depilatory composition comprising an effective amount of a keratin reducing agent.

2. The kit of claim 1, wherein the protective composition comprises from about 1% to about 40% of hydrophobic polymer by weight of the protective composition.

3. The kit of claim 1, wherein the protective composition comprises from about 3% to about 20% of hydrophobic polymer by weight of the protective composition.

4. The kit of claim 1, wherein the hydrophobic polymer is a copolymer, the copolymer comprising at least about 50% by weight of the copolymer of monomers which have hydrophobic side-groups.

5. The kit of claim 1, wherein the hydrophobic polymer is a copolymer, the copolymer comprising about 60% to about 90%, by weight of the copolymer of monomers which have hydrophobic side-groups.

6. The kit of claim 4, wherein each hydrophobic side-group comprises from about 8 to about 40 carbon atoms.

7. The kit of claim 4, wherein the carbon-carbon bonds within the hydrophobic side-groups have a degree of unsaturation of less than about 50%, preferably less than 25% and are more preferably fully saturated.

8. The kit of claim 4, wherein the carbon-carbon bonds within the hydrophobic side-groups are fully saturated.

9. The kit of any of claim 4, wherein the hydrophobic side-groups comprise straight chain alkyl groups.

10. The kit of claim 4, wherein the copolymer comprises monomers having formula (monomer A): 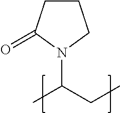
and monomers having formula (monomer B): 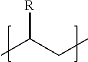
where R is a hydrophobic side-group comprising a straight alkyl chain having from about 12 to about 30 carbon atoms and wherein the copolymer comprises from about 50% to about 99% by weight of monomer B.

11. The kit of claim 4, wherein the copolymer comprises monomers having formula (monomer D): 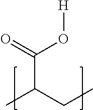
and monomers having formula (monomer E): 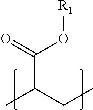
where R₁ is a hydrophobic side-group comprising a straight alkyl chain having from about 10 to about 30 carbon atoms; and monomers having formula (monomer F): 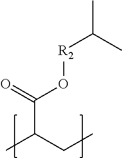
where R₂ is a hydrophobic side-group comprising a straight alkyl chain having from about 4 to about 26 carbon atoms, wherein the copolymer comprises from about 50% to about 99% weight of the combination of monomers E and F.

12. The kit of claim 1, wherein protective composition is an emulsion and the hydrophobic polymer is comprised within the hydrophobic phase thereof.

13. The kit of claim 1, wherein the keratin reducing agent comprises a thioglycolic acid salt, preferably potassium or calcium thioglycolate, or mixtures thereof.

14. The kit of any preceding claim, additionally comprising the following instructions:
(a) An instruction to apply the protective composition over an area of skin to be depilated, preferably an instruction to apply the protective composition over an area which is greater than the area of skin to be depilated;
(b) An instruction to massage the protective composition into the area of skin to be depilated, preferably for at least about 10 seconds, more preferably to massage in a circular motion;
(c) An instruction to apply the depilatory composition to the pre-treated area of skin to be depilated;
(d) An instruction to leave the depilatory composition in place on top of the protective composition for a period of at least about 1 minute;
(e) An instruction to remove both the protective composition and the depilatory composition from the skin, by scraping, wiping or rubbing it off.
(f) An instruction to treat the area of skin which has been depilated with a post-treatment skin care composition.

15. A method of removing hair from skin comprising the steps of:
(a) applying a protective composition according to claim 1 to an area of skin on which unwanted hair is growing.
(b) applying a depilatory composition to the area of skin to which the protective composition has been applied, the depilatory composition comprising a keratin reducing agent.

16. The method of claim 15, comprising the following additional step between step (a) and step (b):
(a1) massaging the protective composition into the skin for at least about 10 seconds.

17. The method of claim 15, comprising the following additional step immediately following step (b):
(c) leaving the depilatory composition in place on the protective composition for a period of at least about 1 minute.
